(19) United States
(12) Patent Application Publication
(10) Pub. No.: US 2011/0288303 A1
(43) Pub. Date: Nov. 24, 2011

(54) Method of Fabricating I-123-IBOX

(75) Inventors: Kang-Wei Chang, Taoyuan County (TW); Shih-Ying Lee, Taoyuan County (TW); Chia-Chieh Chen, Taoyuan County (TW); Lie-Hang Shen, Taoyuan County (TW)

(73) Assignee: ATOMIC ENERGY COUNCIL-INSTITUTE OF NUCLEAR ENERGY RESEARCH, Taoyuan County (TW)

(21) Appl. No.: 12/783,242

(22) Filed: May 19, 2010

(51) Int. Cl. C07D 263/54

(52) U.S. Cl. 548/217

(57) ABSTRACT

A method is provided for fabricating I-123-IBOX. I-123-IBOX is used for SPECT of brain. During the fabrication, radioactivity of reaction vial and purification column can be detected. Thus, I-123-IBOX can be obtained in a short time through a simple process with reduced radioactive harms.

### TECHNICAL FIELD OF THE DISCLOSURE

The present disclosure relates to fabricating I-123-IBOX; more particularly, relates to processing an automatic system for obtaining I-123-IBOX in a short time to be used for emission tomography of brain.

### DESCRIPTION OF THE RELATED ART

More and more people suffer from central nervous system (CNS) disease, especially mental disease like schizophrenia, anxiety, depression, etc., neuron degeneration disease like Alzheimer's disease, and ataxia like Parkinson's disease.

Alzheimer's disease is also called degenerative senile dementia, which is quite normal and is not curable. Older men are apt to fall ill to Alzheimer's disease. 30 to 35 percent of men older than 85 years of age may be ill of that disease. Alzheimer's disease causes involution in fields of cognition, behavior and mentation, where cortex atrophies, brain grooves back widely and ventricle expands. On diagnosing Alzheimer's disease, important indexes include neurofibrillary tangles (NFT) and senile plaque (SP).

In brains of Alzheimer's disease patients, many small round sediments or spots are found. These web-like proteins are amyloid. Hence, finding amyloid beta plaques is a key for diagnosing Alzheimer's disease.

Therefore, amyloid-like tracers are developed for diagnosing Alzheimer's disease; and I-123-IBOX is one of them to be used with microSPECT. Although IMPY may be used, its main function is to diagnose Alzheimer's disease dye like Thioflavine-T. Thus, it is necessary to develop an automatic system for reducing radioactive harms and shortening operating time. Hence, the prior art does not fulfill all users' requests on actual use.

### SUMMARY OF THE DISCLOSURE

The main purpose of the present disclosure is to process an automatic system for obtaining I-123-IBOX to be used for emission tomography of brain while radioactive matter is prevented from leakage.

The second purpose of the present disclosure is to monitor radioactivity of a reaction vial and a purification column and to automatically fabricating short term radiopharmaceuticals within 15 minutes.

The third purpose of the present disclosure is to reduce radioactive harms on fabricating I-123-IBOX yet in a short time with a simple process.

To achieve the above purposes, the present disclosure is a method of fabricating I-123-IBOX, comprising steps of: (a) obtaining hydrogen peroxide (H₂O₂) in a first vial, sodium sulfite (Na₂SO₃) in a second vial, sodium phosphate (Na₂HPO₃) in a third vial, water in a fourth vial and a fifth vial, 50% ethanol in a sixth vial and 95% ethanol in a seventh vial, filling a solution having radioactive ammonium iodide ([¹²³I]NH₄I) into a reaction vial, and adding a precursor of SnIBOX dissolved in ethanol into the reaction vial by using a gas pressure to be mixed with the solution having [¹²³I]NH₄I; (b) processing a pretreatment of washing to a purification column with ethanol and water; (c) mixing acetic acid and vitamin C (ascorbic acid) with 0.9% sodium chloride (NaCl) to be drawn out by an injection needle and then be filled into a first collecting vial through a filter cartridge; (d) turning a sampling valve to a filling position, filling the solution having [¹²³I]NH₄I into a sampling loop by nitrogen gas pressure with slopped part of the solution having [¹²³I]NH₄I flown to a recycle vial, and filling the solution having [¹²³I]NH₄I in the sampling loop into the reaction vial by nitrogen gas pressure to be stirred by gas bubbles with gas in the reaction vial outputted through an activated charcoal column; (e) filling H₂O₂ in the first vial into the reaction vial as an oxygenant to be stirred by gas bubbles; (f) filling Na₂SO₃ in the second vial and Na₂HPO₃ in the third vial into the reaction vial as reductants to be stirred by gas bubbles and ending oxidation after a period of time by stopping stirring; (g) filling solution in the reaction vial into the purification column to process an absorption with a filtered solution flown into a waste vial; (h) filling water in the fourth vial into the reaction vial to be stirred by gas bubbles, filling solution in the reaction vial into the purification column with a filtered solution flown into the waste vial, and filling water in the fifth vial into the purification column to wash with a filtered solution flown into the waste vial; (i) filling 50% ethanol in the sixth vial into the purification column with a filtered solution flown into the waste vial; (j) filling 95% ethanol in the seventh vial into the purification column by nitrogen gas pressure, filling solution in the purification column to an empty vial, and reversely filling solution in the empty vial back to the purification column, and then processing such back-and-forth washing in the above way for several times; (k) filling solution in the purification column into a second collecting vial, and then filling solution in the second collecting vial into a third collecting vial through a filter membrane; (l) refilling 95% ethanol into the seventh vial, replacing the filter membrane with another filter membrane, and replacing the third collecting vial with the first collecting vial obtained in step (c); and (m) filling 95% ethanol in the seventh vial into the purification column by nitrogen gas pressure, filling solution in the purification column into the second collecting vial, filling solution in the second collecting vial into the first collecting vial through the filter membrane to be mixed with the mixed solution of normal saline and vitamin C. Accordingly, a novel method of fabricating I-123-IBOX is obtained.

### BRIEF DESCRIPTION OF THE DRAWINGS

The present disclosure will be better understood from the following detailed description of the preferred embodiment according to the present disclosure, taken in conjunction with the accompanying drawings, in which

FIG. 1 is the flow view showing the preferred embodiment according to the present disclosure; and

FIG. 2 is the view showing the layout of the preferred embodiment.

### DESCRIPTION OF THE PREFERRED EMBODIMENT

The following description of the preferred embodiment is provided to understand the features and the structures of the present disclosure.

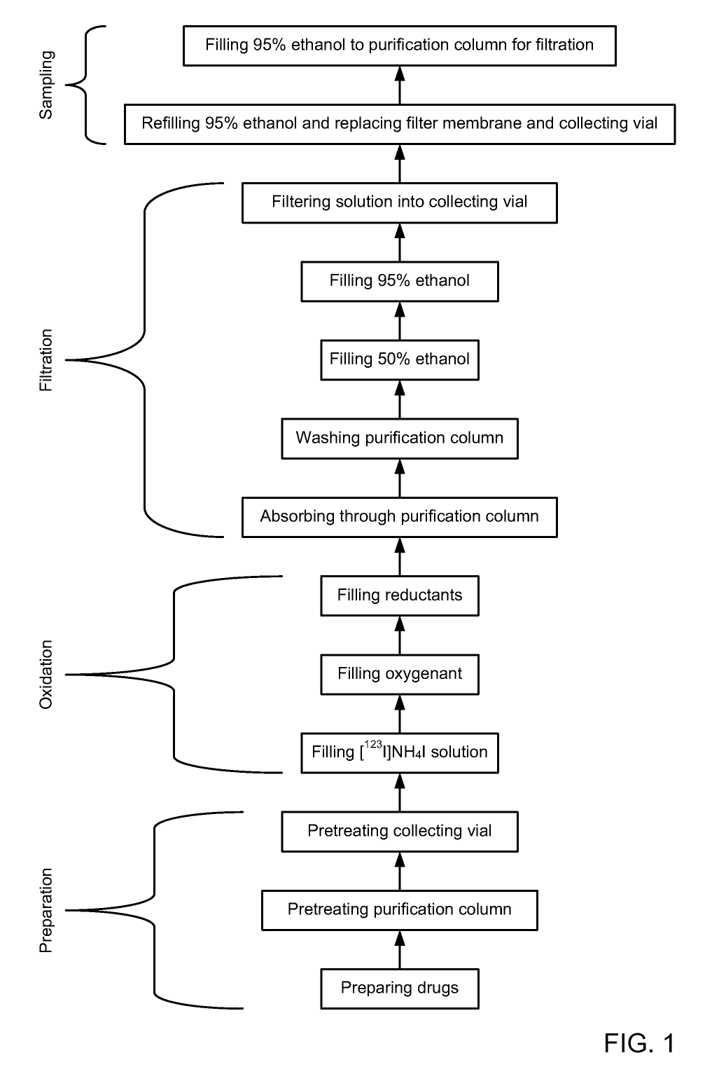

Please refer to FIG. 1, which is a flow view showing a preferred embodiment according to the present disclosure. As shown in the figure, the present disclosure is a method of fabricating I-123-IBOX, comprising the following steps:

Preparation

(a) Preparing drugs 11: A solution having hydrogen peroxide (H₂O₂) is put into a first vial; a solution having sodium sulfite (Na₂SO₃), into a second vial; a solution having sodium phosphate (Na₂HPO₃), a third vial; water, a fourth vial and a fifth vial; a solution having 50% ethanol, a sixth vial; and, a solution having 95% ethanol, a seventh vial. A solution having radioactive ammonium iodide ([¹²³I]NH₄I) is filled into a reaction vial. A precursor of SnIBOX is put into the reaction vial 38 by using a gas pressure to be mixed with the solution having [¹²³I]NH₄I, where the precursor is dissolved into ethanol.

(b) Pretreating purification column 12: A purification column is pretreated, where the purification column is washed with ethanol and water.

(c) Pretreating collecting vial 13: Vitamin C (ascorbic acid) and acetic acid are mixed into a solution having 0.9% sodium chloride (NaCl) to obtain a mixed solution. The mixed solution is drawn out by a sterile injection needle. The sucked mixed solution is filled into a first collecting vial through a filter cartridge.

Oxidation

(d) Filling [¹²³I]NH₄I solution 14: A sampling valve is turned to a filling position. The [¹²³I]NH₄I solution is filled into a sampling loop by nitrogen gas pressure. Slopped part of the [¹²³I]NH₄I solution is flown to a recycle vial. Then, the [¹²³I]NH₄I solution in the sampling loop is filled into the reaction vial by nitrogen gas pressure. The solution in the reaction vial is stirred by gas bubbles formed through the nitrogen gas pressure, where gas in the reaction vial is outputted through an activated charcoal column.

(e) Filling oxygenant 15: H₂O₂ in the first vial is used as an oxygenant to be filled into the reaction vial to be stirred by gas bubbles for reaction.

(f) Filling reductants 16: Na₂SO₃ in the second vial and Na₂HPO₃ in the third vial are filled into the reaction vial as reductants to be stirred by gas bubbles for reaction. Oxidation is ended after a period of time by stopping stirring.

Filtration

(g) Absorbing through purification column 17: The solution in the reaction vial is filled into the purification column for absorption. A filtered solution is flown into the waste vial.

(h) Washing purification column 18: Water in the fourth vial is filled into the reaction vial to be stirred by gas bubbles for reaction. Then, the solution in the reaction vial is filled into the purification column. A filtered solution is flown to the waste vial. Then, water in the fifth vial is filled into the purification column for washing. A filtered solution is flown into the waste vial too.

(i) Filling 50% ethanol 19: 50% ethanol in the sixth vial is filled into the purification column. A filtered solution obtained from the solution in the purification column is flown into the waste vial.

(j) Filling 95% ethanol 20: 95% ethanol in the seventh vial is filled into the purification column. Then, the solution in the purification column is filled into an empty vial. Then, the solution in the empty vial is reversely filled back to the purification column. In this way, the washing is processed back and forth for several times.

(k) Filtering solution into collecting vial 21: The solution in the purification column is filled into a second collecting vial. The solution in the second collecting vial is filled into a third collecting vial through a filter membrane.

Sampling

(l) Refilling 95% ethanol and replacing filter membrane and collecting vial 22: 95% ethanol is refilled into the seventh vial. The filter membrane is replaced with another filter membrane. The third collecting vial is replaced with the first collecting vial obtained in step (c).

(m) Filling 95% ethanol to purification column for filtration 23: 95% ethanol in the seventh vial is filled into the purification column by nitrogen gas pressure. The solution in the purification column is filled into the second collecting vial. The solution in the second collecting vial is filled into the first collecting vial through the filter membrane to be mixed with the prepared mixed solution of normal saline and vitamin C.

Thus, a novel method of fabricating I-123-IBOX is obtained.

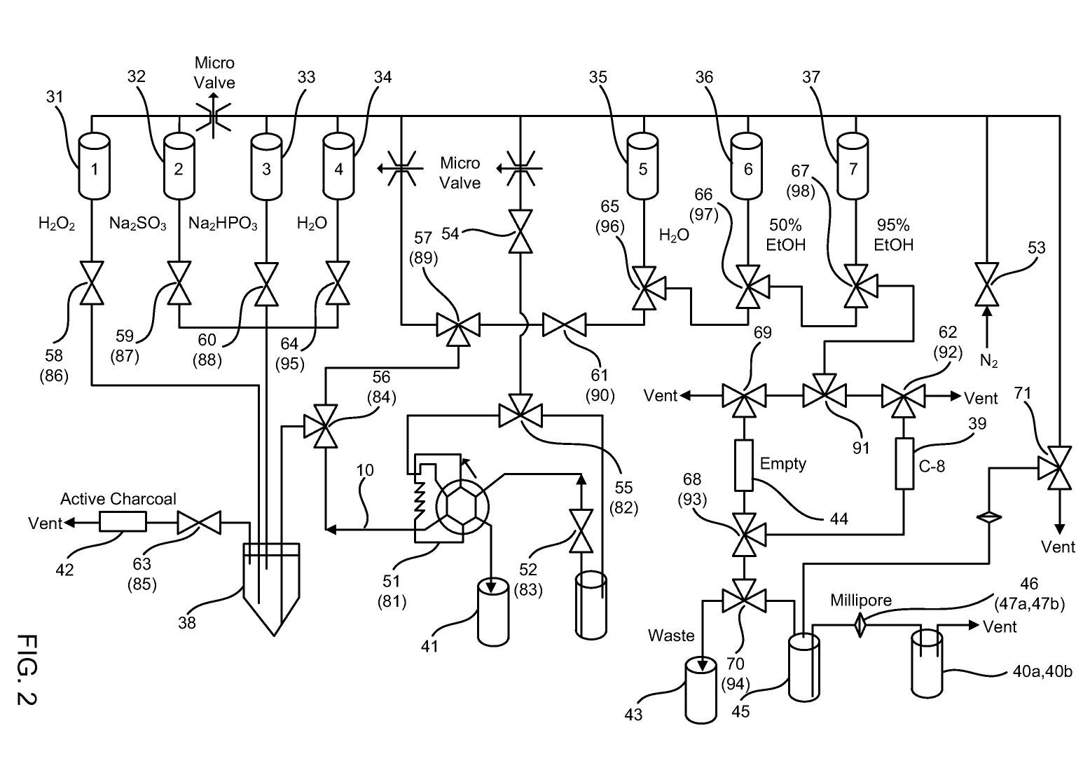

Please refer to FIG. 2, which is a view showing a layout of the preferred embodiment. As shown in the figure, with a 110 volts power supplied, the present disclosure comprises the following steps:

Preparation

(a1) A solution having 5% H₂O₂ is put into a first vial 31; a solution having 300 mg/ml Na₂SO₃, into a second vial 32; a solution having Na₂HPO₃, a third vial 33; water, a fourth vial 34 and a fifth vial 35; a solution having 50% ethanol, a sixth vial 36; and, a solution having 95% ethanol, a seventh vial 37. A solution having [¹²³I]NH₄I is filled into a reaction vial 38, where the solution having [¹²³I]NH₄I has an amount of 300 μl; the solution having [¹²³I]NH₄I has a radioactivity between 150 and 300 mCi; and the reaction vial 38 has a size of 5 ml. A precursor of SnIBOX is put into the reaction vial 38 to be mixed with the solution having [¹²³I]NH₄I, where the precursor has a weight of 100 μg; the precursor is dissolved into ethanol; the precursor is put with a gas pressure; the gas pressure is a pressure of a gas having potassium iodide (KI); and the gas has an amount between 40 and 100 μl.

(b1) A purification column, which is C-8 Sep-pek column, is pretreated, where the purification column is washed with 1 ml ethanol and 5 ml water.

(c1) 75 μl vitamin C and 15 μl acetic acid are mixed into a 20 ml solution having 0.9% NaCl to obtain a mixed solution. 5.5 ml of the mixed solution is drawn out by a sterile injection needle. The drawn-out mixed solution is filled into a first collecting vial 40a through a filter cartridge 46, where the filter cartridge 46 is Millipore Millex GV and has a size of 0.22 μm.

Oxidation

(d1) A first conjunction joint 51 is switched on for turning a control valve 81 (sampling valve) to a filling position. A second conjunction joint 52, a third conjunction joint 53 and a fourth conjunction joint 54 are switched on to fill 0.3 ml of the solution having [¹²³I]NH₄I into a sampling loop 10 through a second control valve 82 and a third control valve 83 by nitrogen gas pressure. The slopped part of the solution having [¹²³I]NH₄I is flown to a recycle vial 41 through the control valve 81. The third conjunction joint 53 is kept switched on until the end of the whole procedure. After the solution having [¹²³I]NH₄I is filled for 3 seconds, the first and the second conjunction joints 51,52 are switched off. A fifth conjunction joint 55 and a sixth conjunction joint 56 are switched on to fill the solution having [¹²³I]NH₄I in the sampling loop 10 into the reaction vial 38 through a fourth control valve 84, where the gas in the reaction vial 38 is drained through a control valve 85 and an activated carbon column 42 having granules of sodium hydroxide (NaOH). After 5 seconds, the fourth, the fifth and the sixth conjunction joints 54,55,56 are switched off. A seventh conjunction joint 57 is switched on after the solution in the reaction vial 38 is stirred for 10 seconds by gas bubbles formed through pressure of a nitrogen gas. Therein, a flow of the nitrogen gas entered into the solution having [¹²³I]NH₄I is controlled by a micro valve to slowly fill the solution having [¹²³I]NH₄I into the sampling loop 10.

(e1) An eighth conjunction joint 58 is switched on. 0.1 ml of the solution having 5% H₂O₂ in the first vial 31 is used as an oxygenant to be filled into the reaction vial 38 through a sixth control valve 86. After 5 seconds, the eighth conjunction joint 58 is switched off. The seventh conjunction joint 57 is switched on. After 5 minutes of stirring by gas bubbles, the seventh conjunction joint 57 is switched off.

(f1) Na₂SO₃ in the second vial 32 and Na₂HPO₃ in the third vial 33 are sued as reductants. At first, a ninth conjunction joint 59 is switched on to fill 1 ml Na₂SO₃ into the reaction vial 38 through a seventh control valve 87. After 5 seconds, the ninth conjunction joint 59 is switched off. The seventh conjunction joint 57 is switched on for stirring by gas bubbles. After 10 seconds, the seventh conjunction joint 57 is switched off. A tenth conjunction joint 60 is switched on to fill 2 ml Na₂HPO₃ into the control valve 38 through an eighth control valve 88. After 5 seconds, the tenth conjunction joint 60 is switched off. The seventh conjunction joint 57 is switched on for stirring by gas bubbles. After 10 seconds, the seventh conjunction joint 57 is switched off and oxidation ends.

Filtration

(g1) The third conjunction joint 53, the tenth conjunction joint 60, an eleventh conjunction joint 61 and a twelfth conjunction joint 62 are opened. A thirteenth conjunction joint 63 is switched on to switch off the fifth control valve 85. The solution in the reaction vial 38 is filled into the purification column 39 through a ninth control valve 89, a tenth control valve 90, an eleventh control valve 91 and a twelfth control valve 92 for absorption. A filtered solution is flown into the waste vial 3 through a thirteenth control valve 93 and a fourteenth control valve 94. After 60 seconds, the eleventh, the twelfth and the thirteenth conjunction joint 61,62,63 are switched off.

(h1) A fourth conjunction joint 64 is switched on. 10 ml water in the fourth vial 34 is filled into the reaction vial 38 through a fifth control valve 95. After 10 seconds, the fourth conjunction joint 64 is switched off. The seventh conjunction joint 57 is switched on for stirring by gas bubbles. After 10 seconds, the seventh conjunction joint 57 is switched off. The third, the eleventh, the twelfth and the fourteenth conjunction joints 53,61,62,64 are switched on. The thirteenth conjunction joint 63 is switched on to switch off the fifth control valve 85. The solution in the reaction vial 38 is fill into the purification column 39 through the ninth, the tenth and the twelfth control valves 89,90,92 for absorption. A filtered solution obtained from the solution in the reaction vial 38 is flown to the waste vial 43 through the thirteenth and the fourteenth control valve 93,94. The eleventh and the thirteenth conjunction joints 61,63 are switched off. After the final product is absorbed in the purification column 39, the twelfth conjunction joint 62 and a fifth conjunction joint 65 are switched on to fill 10 ml water in the fifth vial into the purification column 39 through the twelfth control valve 92 and a sixteenth control valve 96 for obtaining unlabeled I-123 source. A filtered solution obtained from the solution in the purification column 39 is flown into the waste vial 43 through the thirteenth and the fourteenth control valve 93,94. After 300 seconds, the fifteenth conjunction joint 65 is switched off.

(i1) A sixteenth conjunction joint 66 is switched on. 1 ml of 50% ethanol in the sixth vial 36 is filled into the purification column 39 through the eleventh control valve 91, the twelfth control valve 92 and a seventeenth control valve 97 for removing impurities through concentration gradient. A filtered solution obtained from the solution in the purification column 39 is flown into the waste vial 43 through the thirteenth and the fourteenth control valve 93,94. After 30 seconds, the sixteenth conjunction joint 66 is switched off.

(j1) The twelfth conjunction joint 62, a seventeenth conjunction joint 67 and an eighteenth conjunction joint 68 are switched on. 3 ml of 95% ethanol in the seventh vial 37 is filled into the purification column 39 through the eleventh control valve 91, the twelfth control valve 92 and an eighteenth control valve 98. Then, the solution in the purification column 39 is filled into an empty vial 44 through the thirteenth control valve 93. After 30 seconds, the twelfth conjunction joint 62 is switched off. A nineteenth conjunction joint 69 is switched on to make ethanol in the empty vial 44 flow backward. Then, after 30 seconds, the nineteenth conjunction joint 69 is switched off. The twelfth conjunction joint 62 is switched on to make ethanol in the purification column 39 flow back to the empty vial 44. In this way, the washing is processed back and forth for 5 times.

(k1) After 5 times of washing, the eighteenth and the nineteenth conjunction joint 68,69 are switched off. The twelfth conjunction joint 62 and a twentieth conjunction joint 70 are switched on. The solution in the purification column 39 is filled into a second collecting vial 45 through the thirteenth and the fourteenth control valves 93,94. After 30 seconds, the twelfth, the seventeenth and the twentieth conjunction joint 62,67,70 is switched off. A twenty-first conjunction joint is switched on to fill the solution in the second collecting vial 45 into a third collecting vial 40b through a first filter membrane 47a. After 180 seconds, the third and the twenty-first conjunction joints 53,71 are switched off to obtain a product of I-123-IBOX.

Sampling

(l1) 0.9 ml of 95% ethanol is refilled into the seventh vial 37. The first filter membrane 47a is replaced with a second filter membrane 47b. The third collecting vial 40b is replaced with the first collecting vial 40a obtained in step (c1).

(m1) The third, the twelfth, the seventeenth and the twentieth conjunction joints 53,62,67,70 are switched on. 95% ethanol in the seventh vial 37 is filled into the purification column 39 through the eleventh, the twelfth and the eighteenth control valves 91,92,98. Then, the solution in the purification column 39 is filled into the second collecting vial 45 through the thirteenth and the fourteenth control valves 93,94. After 30 seconds, the twelfth, the seventeenth and the twentieth conjunction joints 62,67,70 are switched off. The twenty-first conjunction joint 71 is switched on to fill the product solution in the second collecting vial 45 into the first collecting vial 40a through the second filter membrane 47b to be mixed with the prepared mixed solution of normal saline and vitamin C. After 60 seconds, the third and the twenty-first conjunction joints are switched off.

Through the above steps, a purified I-123-IBOX is obtained after a total process time of 15 minutes, where radioactive matter is prevented from leakage and radioactive harms are reduced. Thus, the whole process can be operated automatically following easy steps with one single reaction vial for fabricating short term radiopharmaceuticals.

To sum up, the present disclosure is a method of fabricating I-123-IBOX, where all reactions are processed in an automatic system for obtaining I-123-IBOX in a short time to be used for emission tomography of brain.

The preferred embodiment herein disclosed is not intended to unnecessarily limit the scope of the disclosure. Therefore, simple modifications or variations belonging to the equivalent of the scope of the claims and the instructions disclosed herein for a patent are all within the scope of the present disclosure.

What is claimed is:

1. A method of fabricating I-123-IBOX, the method comprising:
obtaining hydrogen peroxide (H₂O₂) in a first vial, sodium sulfite (Na₂SO₃) in a second vial, sodium phosphate (Na₂HPO₃) in a third vial, water in a fourth vial and a fifth vial, 50% ethanol in a sixth vial and 95% ethanol in a seventh vial, filling a solution having radioactive ammonium iodide ([¹²³I]NH₄I) into a reaction vial, and adding a precursor of SnIBOX dissolved in ethanol into said reaction vial by using a gas pressure to be mixed with said solution having [¹²³I]NH₄I;
processing a pretreatment of washing to a purification column with ethanol and water;
mixing acetic acid and vitamin C (ascorbic acid) with 0.9% sodium chloride (NaCl) to be drawn out by an injection needle and then be filled into a first collecting vial through a filter cartridge;
turning a sampling valve to a filling position, filling said solution having [¹²³I]NH₄I into a sampling loop by nitrogen gas pressure with slopped part of said solution having [¹²³I]NH₄I flown to a recycle vial, and filling said solution having [¹²³I]NH₄I in said sampling loop into said reaction vial by nitrogen gas pressure to be stirred by gas bubbles with gas in said reaction vial outputted through an activated charcoal column;
filling H₂O₂ in said first vial into said reaction vial as an oxygenant to be stirred by gas bubbles;
filling Na₂SO₃ in said second vial and Na₂HPO₃ in said third vial into said reaction vial as reductants to be stirred by gas bubbles and ending oxidation after a period of time by stopping stirring;
filling solution in said reaction vial into said purification column to process an absorption with a filtered solution flown into a waste vial;
filling water in said fourth vial into said reaction vial to be stirred by gas bubbles, filling solution in said reaction vial into said purification column with a filtered solution flown into said waste vial, and filling water in said fifth vial into said purification column to wash with a filtered solution flown into said waste vial;
filling 50% ethanol in said sixth vial into said purification column with a filtered solution flown into said waste vial;
filling 95% ethanol in said seventh vial into said purification column by nitrogen gas pressure, filling solution in said purification column to an empty vial, and reversely filling solution in said empty vial back to said purification column, and then processing such back-and-forth washing in the above way for several times;
filling solution in said purification column into a second collecting vial, and then filling solution in said second collecting vial into a third collecting vial through a filter membrane;
refilling 95% ethanol into said seventh vial, replacing said filter membrane with another filter membrane, and replacing said third collecting vial with said first collecting vial; and
filling 95% ethanol in said seventh vial into said purification column by nitrogen gas pressure, filling solution in said purification column into said second collecting vial, filling solution in said second collecting vial into said first collecting vial through said filter membrane to be mixed with said mixed solution of normal saline and vitamin C.

2. The method according to claim 1, wherein said gas pressure is a pressure of a gas having potassium iodide (KI).

3. The method according to claim 1, wherein a flow amount of said nitrogen gas is controlled by a micro valve.

4. The method according to claim 1, wherein said solution having [¹²³I]NH₄I is filled for 3 seconds.

5. The method according to claim 1, wherein said nitrogen gas is stirred for 10 seconds.

6. The method according to claim 1, wherein said nitrogen gas is stirred for 5 minutes.

7. The method according to claim 1, wherein said Na₂SO₃ and Na₂HPO₃ are both stirred by nitrogen gas for 10 seconds separately.

8. The method according to claim 1, wherein said nitrogen gas is stirred for 10 seconds.

9. The method according to claim 1, wherein said washing is processed back and forth for 5 times.

10. The method according to claim 1, wherein a total process time of the method is 15 minutes.
